(19) United States
(12) Patent Application Publication
(10) Pub. No.: US 2020/0202975 A1
(43) Pub. Date: Jun. 25, 2020

(54) GENETIC INFORMATION PROCESSING SYSTEM WITH MUTATION ANALYSIS MECHANISM AND METHOD OF OPERATION THEREOF

(71) Applicant: AiOnco, Inc., Menlo Park, CA (US)

(72) Inventor: Gene Lee, Millbrae, CA (US)

(21) Appl. No.: 16/226,380

(22) Filed: Dec. 19, 2018

(51) Int. Cl. G16B 30/00

(52) U.S. Cl. CPC G16B 30/00

(57) ABSTRACT

A genetic information processing system includes: a control unit, configured to: receive an indel analysis tandem repeat k-mer of sequence length k nucleotides from a genome tandem repeat reference catalogue, wherein the indel analysis tandem repeat k-mer is unique within a reference human genome and include: a reference tandem repeat sequence; and flanking sequences directly preceding and following the reference tandem repeat sequence; analyze a DNA sample set, including a healthy sample DNA information and a cancerous sample DNA information, based on the genome tandem repeat reference catalogue including: identify a corresponding healthy sample sequence in the healthy sample DNA information and a corresponding cancerous sample sequence in the cancerous sample DNA information corresponding the indel analysis tandem repeat k-mer; determine whether the corresponding cancerous sample sequence includes a tumorous indel mutation based on a comparison between the corresponding cancerous sample sequence and the corresponding healthy sample sequence; and modify the genome tandem repeat reference catalogue to identify the reference tandem repeat sequence of the instance of indel analysis tandem repeat k-mer as a tumor marker when the tumorous indel mutation exists in the corresponding cancerous sample sequence.

### TECHNICAL FIELD

An embodiment of the present invention relates generally to a genetic information processing system, and more particularly to a system for mutation analysis.

### BACKGROUND

Modern consumer and industrial electronics, especially devices such as personal medical devices, cellular phones, and portable diagnostic devices, are providing increasing levels of functionality to support modern life, including evaluation and diagnosis of bodily ailments and diseases. Research and development in the existing technologies can take a myriad of different directions.

As users become more empowered with the growth of personal medical devices and portable diagnostic devices, new and old paradigms begin to take advantage of this new device space for on demand health diagnostics. There are many technological solutions to take advantage of this new device capability for on demand health diagnostics. However, users are often not provided with the ability to analyze genetic material for the development of mutations and tumors.

Thus, a need still remains for a genetic information processing system with a mutation analysis mechanism. In view of the ever-increasing commercial competitive pressures, along with growing consumer expectations and the diminishing opportunities for meaningful product differentiation in the marketplace, it is increasingly critical that answers be found to these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### SUMMARY

An embodiment of the present invention provides a genetic information processing system, including: a control unit configured to: a control unit configured to: receive an indel analysis tandem repeat k-mer of sequence length-k nucleotides from a genome tandem repeat reference catalogue, wherein the indel analysis tandem repeat k-mer is unique within a reference human genome and include: a reference tandem repeat sequence; and flanking sequences directly preceding and following the reference tandem repeat sequence; analyze a DNA sample set, including a healthy sample DNA information and a cancerous sample DNA information, based on the genome tandem repeat reference catalogue including: identify a corresponding healthy sample sequence in the healthy sample DNA information and a corresponding cancerous sample sequence in the cancerous sample DNA information corresponding the indel analysis tandem repeat k-mer; determine whether the corresponding cancerous sample sequence includes a tumorous indel mutation based on a comparison between the corresponding cancerous sample sequence and the corresponding healthy sample sequence; and modify the genome tandem repeat reference catalogue to identify the reference tandem repeat sequence of the instance of indel analysis tandem repeat k-mer as a tumor marker when the tumorous indel mutation exists in the corresponding cancerous sample sequence.

An embodiment of the present invention provides a method of operation of a genetic information processing system including: receiving an indel analysis tandem repeat k-mer of sequence length-k nucleotides a genome tandem repeat reference catalogue, wherein the indel analysis tandem repeat k-mer is unique within a reference human genome and include: a reference tandem repeat sequence; and flanking sequences directly preceding and following the reference tandem repeat sequence; analyzing a DNA sample set, including a healthy sample DNA information and a cancerous sample DNA information, based on the genome tandem repeat reference catalogue including: identify a corresponding healthy sample sequence in the healthy sample DNA information and a corresponding cancerous sample sequence in the cancerous sample DNA information corresponding the indel analysis tandem repeat k-mer; determine whether the corresponding cancerous sample sequence includes a tumorous indel mutation based on a comparison between the corresponding cancerous sample sequence and the corresponding healthy sample sequence; and modifying the genome tandem repeat reference catalogue to identify the reference tandem repeat sequence of the instance of indel analysis tandem repeat k-mer as a tumor marker when the tumorous indel mutation exists in the corresponding cancerous sample sequence.

An embodiment of the present invention provides a non-transitory computer readable medium including instructions executable by a control circuit for a genetic information processing system, the instructions including: receiving an indel analysis tandem repeat k-mer of sequence length-k nucleotides a genome tandem repeat reference catalogue, wherein the indel analysis tandem repeat k-mer is unique within a reference human genome and include: a reference tandem repeat sequence; and flanking sequences directly preceding and following the reference tandem repeat sequence; analyzing a DNA sample set, including a healthy sample DNA information and a cancerous sample DNA information, based on the genome tandem repeat reference catalogue including: identify a corresponding healthy sample sequence in the healthy sample DNA information and a corresponding cancerous sample sequence in the cancerous sample DNA information corresponding the indel analysis tandem repeat k-mer; determine whether the corresponding cancerous sample sequence includes a tumorous indel mutation based on a comparison between the corresponding cancerous sample sequence and the corresponding healthy sample sequence; and modifying the genome tandem repeat reference catalogue to identify the reference tandem repeat sequence of the instance of indel analysis tandem repeat k-mer as a tumor marker when the tumorous indel mutation exists in the corresponding cancerous sample sequence.

Certain embodiments of the invention have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

FIG. 1 is a genetic information processing system 100 with a mutation analysis mechanism in an embodiment of the present invention.

FIG. 2 is a characterization of a unique reference tandem repeat k-mer for the genome tandem repeat reference catalogue of FIG. 1.

FIG. 3 is an example of the unique reference tandem repeat k-mers of the genome tandem repeat reference catalogue of FIG. 1.

FIG. 4 is an example illustration of an entry in the genome tandem repeat reference catalogue.

FIG. 5 is an exemplary block diagram of the genetic information processing system.

FIG. 6 is a control flow for the functions of the genetic material analysis system.

FIG. 7 is a flow chart of a method of operation of the genetic information processing system in an embodiment of the present invention.

### DETAILED DESCRIPTION

The following embodiments are described in sufficient detail to enable those skilled in the art to make and use the invention. It is to be understood that other embodiments would be evident based on the present disclosure, and that system, process, or mechanical changes may be made without departing from the scope of an embodiment of the present invention.

In the following description, numerous specific details are given to provide a thorough understanding of the invention. However, it will be apparent that the invention may be practiced without these specific details. In order to avoid obscuring an embodiment of the present invention, some well-known system configurations, and process steps are not disclosed in detail.

The drawings showing embodiments of the system are semi-diagrammatic, and not to scale and, particularly, some of the dimensions are for the clarity of presentation and are shown exaggerated in the drawing figures.

The term “module” referred to herein can include software, hardware, or a combination thereof in an embodiment of the present invention in accordance with the context in which the term is used. For example, the software can be machine code, firmware, embedded code, and application software. Also for example, the hardware can be circuitry, processor, computer, integrated circuit, integrated circuit cores, a pressure sensor, an inertial sensor, a microelectromechanical system (MEMS), passive devices, or a combination thereof. Further, if a module is written in the apparatus claims section below, the modules are deemed to include hardware circuitry for the purposes and the scope of apparatus claims.

The modules in the following description of the embodiments can be coupled to one other as described or as shown. The coupling can be direct or indirect without or with, respectively, intervening items between coupled items. The coupling can be physical contact or by communication between items.

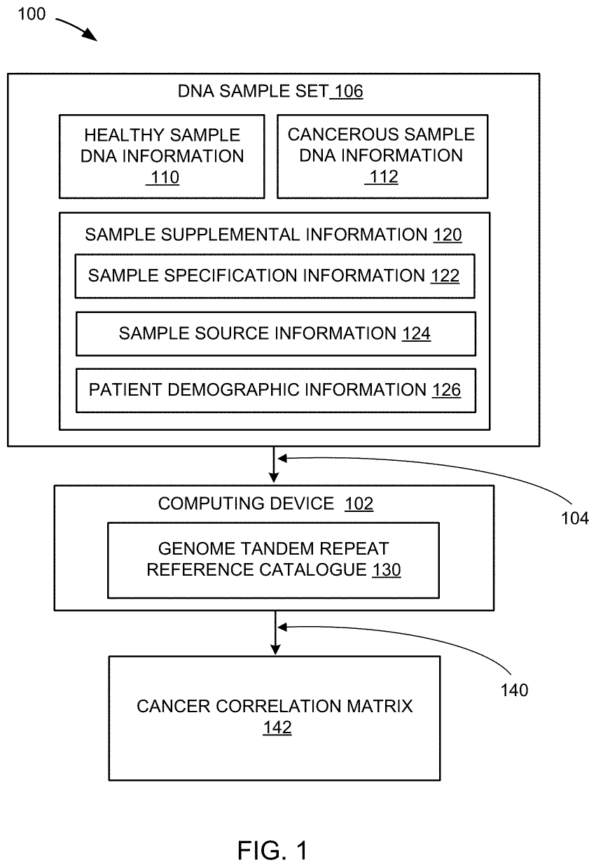

Referring now to FIG. 1, therein is shown a genetic information processing system 100 with a mutation analysis mechanism in an embodiment of the present invention. The mutation analysis mechanism is a mechanism to identify and analyze mutations in genetic information representing genetic material, such as sequenced Deoxyribonucleic Acid (hereinafter “DNA”) segments. For example, the mutation analysis mechanism can identify mutations and determine the existence of tumorous DNA sequences.

The genetic information processing system 100 can include a computing device 102 for processing the genetic information. For example, the computing device 102 can be any of a variety or type of computing devices, such as a notebook or laptop computer, a multimedia computer, a desktop computer, grid-computing resources, a virtualized computer resource, cloud computing resource, peer-to-peer distributed computing devices, a DNA sequencing device, or a combination thereof. Details of the computing device 102 will be described below.

The genetic information processing system 100 can receive a system input 104. The system input 104 is information for processing by the computing device 102. For example, the system input 104 can be a DNA sample set 106, which is a set of sequenced DNA information. Examples of the DNA sample set 106 can include genetic information derived or extracted from human patients, such as tissue extracted during a biopsy or from cell free DNA, which refers to DNA that is not encapsulated within a cell, in bodily fluids. The DNA sample set 106 can be in the form of coded or un-coded text strings that represent the DNA sequences.

The DNA sample set 106 can include healthy sample DNA information 110, and cancerous sample DNA information 112. The healthy sample DNA information 110 is sequenced DNA derived from biological samples that are free of cancer. The cancerous sample DNA information 112 is sequenced DNA derived from biological samples with a confirmed case of a particular form of cancer. In general, the healthy sample DNA information 110 and the cancerous sample DNA information 112 for a particular instance of the DNA sample set 106 can be samples taken from a single human patient.

Both the healthy sample DNA information 110 and the cancerous sample DNA information 112 can include sample supplemental information 120. The sample supplemental information 120 is information that characterizes various aspects of the healthy sample DNA information 110 and cancerous sample DNA information 112. For example, the sample supplemental information 120 can include information such as sample specification information 122, sample source information 124, patient demographic information 126, or a combination thereof.

The sample specification information 122 is technical information or specifications about the sequenced DNA within the DNA sample set 106. For example, the sample specification information 122 can include information about the location within the genome to which the DNA fragments correspond, such as intron and exon regions, specific genes, or chromosomes; the process, methods, and instrumentation used to extract and sequence the genetic material; the number of sequencing reads for each sample, the read length for each of the sequence reads, or a combination thereof.

The sample source information 124 can be details about origin of the sample information. For example, the sample source information 124 can include information about the cancer type, the stage of cancer development, organ or tissue form which the sample was extracted, or a combination thereof.

The patient demographic information 126 is demographic information about the patient from which the sample was taken. For example, the patient demographic information 126 can include the age, the gender, the ethnicity, geographic location of where the patient resides or has been, the duration of time the patient stayed or resided at the geographic location, predispositions for genetic disorders or cancer development, or a combination thereof.

In an embodiment of the genetic information processing system 100, the DNA sample set 106 can be analyzed with the mutation analysis mechanism to identify mutation patterns in specific DNA sequences that can be used as markers to determine the existence of a particular form of cancer or the possibility that cancer will develop. For example, the genetic information processing system 100 can identify the mutation patterns based on differences between specific sequences in the healthy sample DNA information 110 and the cancerous sample DNA information 112 that both correspond to the same location within the human genome based on a genome tandem repeat reference catalogue 130.

The genome tandem repeat reference catalogue 130 is a catalogue of tandem repeat sequences within a human genome that can be uniquely identified. As an example, the genome tandem repeat reference catalogue 130 can be based on a reference genome, such as the GRCh38 reference genome. The tandem repeat sequences are DNA sequences that include a series of multiple instances of directly adjacent identical repeating nucleotide units, such as microsatellite DNA sequences. The genetic information processing system 100 can use the uniquely identifiable tandem repeat sequences of the genome tandem repeat reference catalogue 130 as reference sequences to identify corresponding sequences in the healthy sample DNA information 110 and cancerous sample DNA information 112. The corresponding sequences in the healthy sample DNA information 110 and cancerous sample DNA information 112 can be analyzed with the mutation analysis mechanism to identify mutated sequences and determine whether the identified mutations in the cancerous sample DNA information 112 are tumorous. The genetic information processing system 100 can use the information from the mutation analysis mechanism, such as the tumorous sequences identified in the cancerous sample DNA information 112, and the sample supplemental information 120 to modify or supplement entries for the tandem repeat sequences in the genome tandem repeat reference catalogue 130. Details of the mutation analysis mechanism will be discussed below.

In an embodiment of the invention, the genetic information processing system 100 can generate a system output 140, such as a cancer correlation matrix 142, from the genome tandem repeat reference catalogue 130. The cancer correlation matrix 142 is a matrix that correlates identified tumorous sequence to specific types of cancer. For example, the cancer correlation matrix 142 can be an index that includes multiple instances of the uniquely identifiable tandem repeat sequences in the genome tandem repeat reference catalogue 130 that, when found to tumorous, indicate the existence of a particular form of cancer or the possibility that a particular form of cancer will develop. Details regarding generation of the cancer correlation matrix 142 will be discussed below.

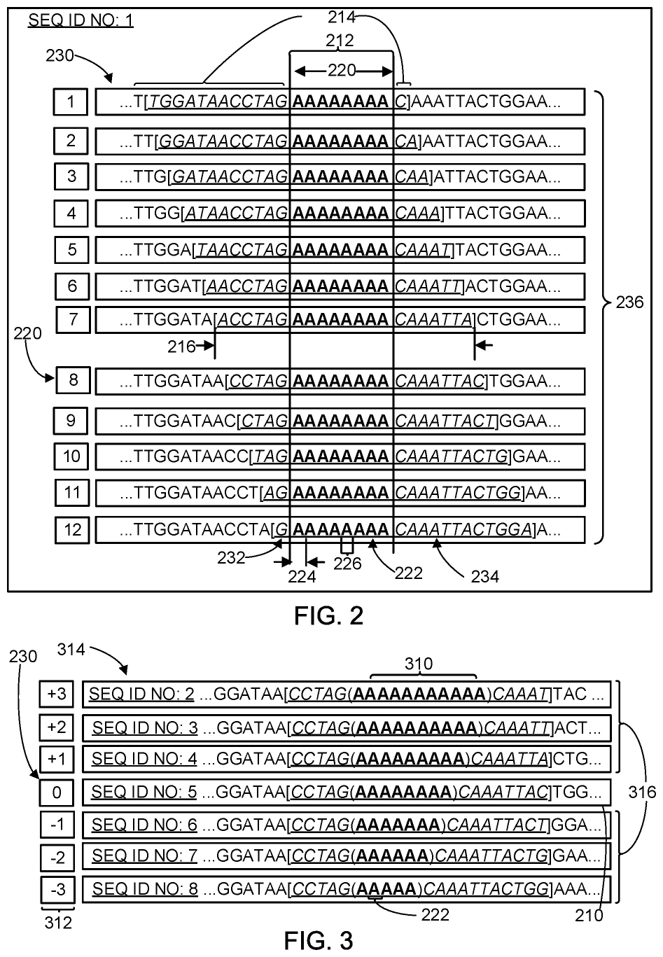

Referring now to FIG. 2, therein is shown a characterization of a unique reference tandem repeat k-mer 210 for the genome tandem repeat reference catalogue 130 of FIG. 1. The unique reference tandem repeat k-mer 210 is a DNA sequence that appears only once within the reference human genome. The unique reference tandem repeat k-mer 210 can be identified based on various characteristics, including a reference tandem repeat sequence 212, flanking sequences 214, and a sequence length k 216.

The sequence length k 216 defines the total number of base pairs in the unique reference tandem repeat k-mer 210 as the value “k”. The term base pairs refer to the nucleotides in DNA of Adenine (A), Cytosine (C), Guanine (G), thymine (T). For illustrative purposes, FIG. 2 depicts the unique reference tandem repeat k-mer 210 with the sequence length k 216 of 21 base pairs, although it is understood that the sequence length k 216 for the unique reference tandem repeat k-mer 210 can be different. For example, the sequence length-k 216 can be greater than or less than 21 base pairs. As a specific example, the sequence length k 216 can be in a range of base pairs from 19 base pairs to 50 or more base pairs.

The reference tandem repeat sequence 212 is a DNA sequence, of a specified minimum length, that is a series of multiple instances of directly adjacent identical repeating nucleotide units. For example, the reference tandem repeat sequence 212 can be a minisatellite DNA or microsatellite DNA sequence of a specified minimum length. Each instance of the reference tandem repeat sequence 212 can be characterized by a tandem repeat sequence length 220, which is the total length of or total number of nucleotide base pairs in the sequence, and a reference repeat unit 222. For illustrative purposes, FIG. 2 illustrates a specific instance for the reference tandem repeat sequence 212 of “AAAAAAAA”, annotated as “A8”, located at the molecular position starting at “10,513,372” on chromosome 22. In this example, the reference tandem repeat sequence 212 of FIG. 2 includes the tandem repeat sequence length 220 of 8 base pairs.

The reference repeat unit 222 is a single unit of the repeating nucleotide pattern in the reference tandem repeat sequence 212. The reference repeat unit 222 can be characterized by a repeat unit length 224 and a repeat unit pattern 226. The repeat unit length 224 is the number of nucleotides within the reference repeat unit 222. The repeat unit pattern 226 is the combination of base pairs that form the reference repeat unit 222. For example, the repeat unit length 224 can be a mono-nucleotide; a di-nucleotide including the repeat unit pattern 226 of a combination of two different nucleotides; a tri-nucleotide including the repeat unit pattern 226 of a combination of two or three nucleotides; or a tetra-nucleotide including the repeat unit pattern 226 of a combination of two, three, or four different nucleotides. FIG. 2 illustrates the reference repeat unit 222 with repeat unit length 224 of 1 base pair and the repeat unit pattern 226 of the nucleotide “A”.

It has been found that detection of mutations in DNA sequences is facilitated by the repeating patterns of the reference repeat unit 222 in the reference tandem repeat sequence 212. For example, changes to the pattern of the reference repeat unit 222 through substitution mutations or number of the reference repeat unit 222 can be more readily detected due to the consistent repetitive nature of the reference repeat unit 222. Thus, the reference tandem repeat sequence 212 is used to improve detection of mutations.

Each instance of the reference tandem repeat sequence 212 can be selected as a subset of the microsatellites or tandem repeat sequences within the reference genome, generally referred to hereinafter as genome tandem repeat sequences. More specifically, the reference tandem repeat sequence 212 can be selected based on the tandem repeat sequence length 220. For example, the reference tandem repeat sequence 212 can be selected as the genome tandem repeat sequence with the tandem repeat sequence length 220 that exceed a minimum number of base pairs. For example, the reference tandem repeat sequence 212 can be selected as the genome tandem repeat sequence with the tandem repeat sequence length 220 having the minimum number of base pairs ranging between 5 base pairs and 8 base pairs. In other words, the reference tandem repeat sequence 212 can be a sequence of 5 or more base pairs, 6 or more base pairs, 7 or more base pairs, or 8 or more base pairs.

It has been found that the probability of mutation occurrences decreases as the tandem repeat sequence length 220 is reduced. In particular, the mutation rate for the tandem repeat sequence length 220 of less than five base pairs is significantly less than the genome tandem repeat sequences with the tandem repeat sequence length 220 of five or more base pairs. Thus, the reference tandem repeat sequence 212 can be selected as the genome tandem repeat sequence with the tandem repeat sequence length 220 of five or greater.

Each instance of the reference tandem repeat sequence 212 can be included in or as part of a sequence with the sequence length k 216, herein referred to as tandem repeat associated k-mers 230. More specifically, the tandem repeat associated k-mers 230 are a set of sequence variations with the sequence length k 216 that include a specific one of the reference tandem repeat sequence 212.

The variations represented by the tandem repeat associated k-mers 230 can be determined by the flanking sequences 214. The flanking sequences 214 are the base pairs that both immediately precede and immediately follow the reference tandem repeat sequence 212 within the reference genome. More specifically, the flanking sequences 214 are the specific instances of base pairs that exist immediately preceding and immediately following the reference tandem repeat sequence 212 at a specific location within the reference human genome. The flanking sequences 214 that precede the reference tandem repeat sequence 212 can be referred to as a leading flanking sequence 232 and the flanking sequences 214 that follow the reference tandem repeat sequence 212 can be referred to as a tailing flanking sequence 234. The leading flanking sequence 232 and the tailing flanking sequence 234 include at least one base pair and are not part of the reference tandem repeat sequence 212. The flanking sequences 214 are illustrated in FIG. 2 by the italicized characters.

The total number of base pairs in the leading flanking sequence 232 and the tailing flanking sequence 234, referred to as the flanking sequence sum, is a fixed value based on the sequence length k 216 and the tandem repeat sequence length 220. The flanking sequence sum can be calculated as the difference between the sequence length k 216 of the unique reference tandem repeat k-mer 210 or the tandem repeat associated k-mers 230 and the tandem repeat sequence length 220 of the reference tandem repeat sequence 212. As an example, for one of the tandem repeat associated k-mers 230 having the sequence length k 216 of 21 base pairs and a tandem repeat sequence length 220 of 8 base pairs, the flanking sequence sum is 13 base pairs.

Each of the tandem repeat associated k-mers 230 can represent one of a number of position variant k-mers 236 based on the flanking sequences 214. The position variant k-mers 236 are specific instances of the tandem repeat associated k-mers 230 with specific numbers of base pairs in the leading flanking sequence 232 and the tailing flanking sequence 234. For example, each of the position variant k-mers 236 can differ from one another according to the number of base pairs included in the leading flanking sequence 232 and the tailing flanking sequence 234. In general, the number of base pairs included in the leading flanking sequence 232 and the tailing flanking sequence 234 can vary inversely between the different instances of the position variant k-mers. The position variant k-mers 236 are illustrated in FIG. 2 as the sequence of base pairs within the brackets.

As an example, the each of the position variant k-mers 236 illustrated in FIG. 2 has the sequence length k 216 of 21 base pairs and the tandem repeat sequence length 220 of 8 base pairs. To continue the example, a first instance of the position variant k-mer 236 can have the leading flanking sequence 232 of 12 base pairs and the tailing flanking sequence 234 of 1 base pair; a second instance of the position variant k-mer 236 with the leading flanking sequence 232 having 11 base pairs and the tailing flanking sequence 234 having 2 base pairs; and so on until the last instance of the position variant k-mers 236, which includes the leading flanking sequence 232 having 1 base pair and the tailing flanking sequence 234 having 12 base pairs.

The total number of the position variant k-mers 236, referred to as a position variant total, for a given k-mer can be calculated as:

position variant total=(sequence length k)−(tandem repeat sequence length)−1

For this example, the instance of the tandem repeat associated k-mers 230 illustrated in FIG. 2 can have the position variant total of 12, representing 12 different instances of the position variant k-mers 236 for the sequence length k 216 of 21 and the tandem repeat sequence length 220 of 6.

The tandem repeat associated k-mers 230 for a particular instance of the reference tandem repeat sequence 212 can be determined as one of the unique reference tandem repeat k-mers 210 when one or more of the position variant k-mers 236 is found to be unique within the reference genome that is used as the basis for the genome tandem repeat reference catalogue 130. More specifically, the position variant k-mers 236 that only appears once or exists in only one position within the reference genome can be identified as one of the unique reference tandem repeat k-mers 210.

It has been found that the combination of reference tandem repeat sequence 212 and the flanking sequences 214 of the unique reference tandem repeat k-mer 210 can enable accurate and precise identification of corresponding sequences in the healthy sample DNA information 110 of FIG. 1, the cancerous sample DNA information 112 of FIG. 1, or a combination thereof, both of which include the same instance of the reference tandem repeat sequence 212 from the unique reference tandem repeat k-mer 210. Since a particular sequences that share the same instance of the rerepeat unit pattern 226 and the repeat unit length 224 can exist in numerous locations within the human genome, using the reference tandem repeat sequences 212 alone as a basis for searching or matching can lead to misidentification and inaccurate results when attempting to identify a specific instance of the reference tandem repeat sequence 212 that exists at a specific location within the human genome. For example, conducting a search through the healthy sample DNA information 110, the cancerous sample DNA information 112, or a combination thereof for a sequence match to a specific instance of the reference tandem repeat sequence 212 alone can potentially return numerous instances of the same tandem repeat sequence without any way to distinguish the sequence location of one from the other. As a specific example, a search for a text string representing a particular instance of the reference tandem repeat sequence 212 can return an inflated or inaccurate count of matching strings in the healthy sample DNA information 110, the cancerous sample DNA information 112, or a combination thereof which can be difficult or impossible to parse for location information of the sequences. For instance, within chromosome 22 alone, the reference tandem sequence 212 of “A8” appears at least 26 times at various locations. Thus, because the combination of reference tandem repeat sequences 212 and the flanking sequences 214 of the unique reference tandem repeat k-mer 210 can be precisely located within the genome, the unique reference tandem repeat k-mer 210 provide the benefit of being used to identify corresponding sequences in the healthy sample DNA information 110, the cancerous sample DNA information 112, or a combination thereof.

Referring now to FIG. 3, therein is shown an example of a single instance of the tandem repeat associated k-mers 230 for one instance of the reference tandem repeat sequence 212 in the genome tandem repeat reference catalogue 130 of FIG. 1. The example of the reference tandem repeat sequence 212 is shown in conjunction with a number of tandem repeat indel variants 310. The tandem repeat indel variants 310 are variations of the reference tandem repeat sequence 212 that include changes in the number of the reference repeat unit 222 (which are illustrated by the sequences within the parenthesis). More specifically, the tandem repeat indel variants 310 are instances of the reference tandem repeat sequence 212 that include insertions or deletions of one or more of the reference repeat unit 222 in the reference tandem repeat sequence 212. As an example, the reference tandem repeat sequence 212 of “AAAAAAAA” beginning at position 10,513,372 on chromosome 22 is used for illustrative purposes. For the sake of brevity, the reference tandem repeat sequence 212 and the tandem repeat indel variants 310 will be annotated with the repeat unit pattern 226 of FIG. 2 and the number of repeat units in either the reference tandem repeat sequence 212 or the tandem repeat indel variants 310. For example, “AAAAAAAA” will be referred to as “A8” since the repeat unit pattern 226 is “A” and the reference tandem repeat sequence 212 includes eight of the reference repeat unit 222 of FIG. 2. Examples of the tandem repeat indel variants 310 illustrated in FIG. 2 show insertions to the reference tandem repeat sequence 212 as “A9”, “A10”, and “A11” while the deletions are shown as “A7”,” “A6”, and “A5”. The tandem repeat indel variants 310 can represent insertion mutations and deletion mutations, hereinafter referred to as indel mutations, relative to the reference tandem repeat sequence 212.

The number of the tandem repeat indel variants 310 associated with the reference tandem repeat sequence 212 can be determined by an indel variant value 312. The indel variant value 312 is an integer value that represents the number of insertions and deletions of the reference repeat unit 222 to the reference tandem repeat sequence 212 for the tandem repeat indel variants 310. For example, negative integer values of the indel variant value 312 can represent deletions of the reference repeat unit 222, positive integer values of the indel variant value 312 can represent insertions of the reference repeat unit 222, and the indel variant value 312 of zero can correspond to the reference tandem repeat sequence 212 as it exists within the human genome, that is, without either insertion or deletions.

Each of the tandem repeat indel variants 310 can be included in associated tandem repeat indel k-mers 316. The associated tandem repeat indel k-mers 316 are sequences of the sequence length k 216 of FIG. 2 including an instance of the reference tandem repeat sequence 212 that exists at a specific location in the reference genome, but with insertions or deletions of one or more of the reference repeat unit 222. In other words, the associated tandem repeat indel k-mers 216 is a sequence that replaces the reference tandem repeat sequence 212 at a specific location in the human genome with one of the tandem repeat indel variants 310. As an example, for the reference tandem repeat sequence 212 “A8” beginning at position 10,513,372 on chromosome 22, the associated tandem repeat indel k-mers 216 preserves the existing base pairs that precede and follow the particular instance of the reference tandem repeat sequence 212 “A8” as the flanking sequences 230, but can replace the reference tandem repeat sequences 212 with one of the tandem repeat indel variants 310. Similar to the tandem repeat associated k-mers 230, the associated tandem repeat indel k-mers 316 can include the leading flanking sequence 232 of FIG. 2 and the tailing flanking sequence 234 of FIG. 2, where the leading flanking sequence 232 and the tailing flanking sequence 234 include at least one base pair and are not part of the tandem repeat indel variants 310. For example, an instance of the associated tandem repeat indel k-mers 316 based on the unique reference tandem repeat k-mer 210 with the leading flanking sequence 232 of “CCTAG” and the tailing flanking sequence 234 of “CAATTAC” can replace the reference tandem repeat sequence 212 of “A8” with one of the tandem repeat indel variants 310. As specific examples, as illustrated in FIG. 3, the reference tandem repeat sequence 212 “A8” can be replaced with “A11”, “A10”, or “A9” corresponding to the indel variant value 312 of “+3”, “+2”, and “+1”, respectively, which represent insertions of the reference repeat unit 222. To continue the specific example, the reference tandem repeat sequence 212 “A8” can be replaced with “A5”, “A6”, or “A7” corresponding to the indel variant value 312 of “−3”, “−2”, and “−1”, respectively, which represent insertions of the reference repeat unit 222.

In general, for a given instance of the reference tandem repeat sequence 212, the associated tandem repeat indel k-mers 316 that include the tandem repeat indel variants 310 are of the same value of the sequence length k 216 as the unique reference tandem repeat k-mer 210 of FIG. 2 or the tandem repeat associated k-mers 230 that include the particular instance of the reference tandem repeat sequence 212 that is replaced by the tandem repeat indel variants 310. For example, as illustrated in FIG. 3, the tandem repeat associated k-mers 230 with the sequence length k 216 of 21 base pairs for the reference tandem repeat sequence 212 “A8” beginning at position 10,513,372 on chromosome 22 will have the associated tandem repeat indel k-mers 316 with the sequence length k 216 of 21 base pairs, regardless of the number of base pairs in the tandem repeat indel variants 310. As specific examples, the associated tandem repeat indel k-mers 316 of “A5” and “A13” will have a total number of base pairs in the flanking sequences 214 of 16 and 10, respectively.

The associated tandem repeat indel k-mers 316 can be similar to the tandem repeat associated k-mers 230 in that the associated tandem repeat indel k-mers 216 are a set of sequence variations with the sequence length k 216 that include the position variant k-mers 236 of FIG. 2 that include the tandem repeat indel variants 310. More specifically, each of the position variant k-mers 236 for the associated tandem repeat indel k-mers 216 can include a specific numbers of base pairs in the leading flanking sequence 232 and the tailing flanking sequence 234 for a given instance of the tandem repeat indel variants 310. For example, each of the position variant k-mers 236 can differ from one another according to the number of base pairs included in the leading flanking sequence 232 and the tailing flanking sequence 234. In general, the number of base pairs included in leading flanking sequence 232 and the tailing flanking sequence 234 can vary inversely between the different instances of the position variant k-mers. The total number of the associated tandem repeat indel k-mers 316, referred to as an indel position variant total, for a specific value for the sequence length k 216 can be calculated as:

IPVT=(k)−(TRSL+IVV)−1

where “IPVT” represents the indel position variant total, “k” represents the sequence length k 216, “TRSL” represents the tandem repeat sequence length 220, and “IVV” represents the indel variant value 312. In general, the indel position variant total can vary depending on the indel variant value 312 that represents one of the tandem repeat indel variant 310. As examples, for the reference tandem repeat sequence 212 of “A8” and the sequence length k 216 of 21, the indel position variant totals for the associated tandem repeat indel variant k-mers 316 that includes the tandem repeat indel variants k-mers 210 of “A5” and “A11” are 15 and 9, respectively. In the example of the associated tandem repeat indel variant k-mers 316 that includes the tandem repeat indel variants k-mers 210 of “A5”, the 1st instance of the position variant k-mers 236 can include 15 base pairs in the leading flanking sequence 232 and 1 base pair in the tailing flanking sequence 234, while the 15th instance of the position variant k-mers 235 can include 1 base pair in the leading flanking sequence 232 and 15 base pairs in the tailing flanking sequence 234. For the sake of brevity, only one instance of the position variant k-mers 236 for each of the tandem repeat indel variants 310 is illustrated in FIG. 3.

In general, the indel variant value 312 can be selected to maximize the number of possible insertions and deletions that can occur in the reference tandem repeat sequences 212. However, the indel variant value 312 that is too high can reduce the number of possible sequences that can be used in by the mutation analysis mechanism. For example, as the total number of base pairs in the tandem repeat indel variant approaches the sequence length k 216, fewer of the associated tandem repeat indel k-mers 316 are possible. Thus, it has been found that the indel variant value 312 in the range of 3 to 5 can provide sufficient coverage for varying degrees of possible insertion and deletion mutations in the cancerous sample DNA information 112 and also cover possible variations in the healthy sample DNA information 110 relative to the unique reference tandem repeat k-mers 210. For illustrative purposes, the unique reference tandem repeat sequence 212 in FIG. 3 is shown with the tandem repeat indel variants 310 with the indel variant value 312 of ranging between −3 to +3, which corresponds to 3 deletions or 3 insertions, respectively, of the reference repeat unit 222 in the reference tandem repeat sequence 212. The tandem repeat indel variants 310 with the indel variant value 312 of zero correspond to a sequence with no insertions or deletions and represents the reference tandem repeat sequences 212.

The tandem repeat indel variants 310, along with the unique reference tandem repeat k-mers 210 of FIG. 2, can be used to identify indel mutations in the cancerous sample DNA information 112. For example, the genetic information processing system 100 of FIG. 1 can use the tandem repeat indel variant 310 of one instance of the unique reference tandem repeat sequence 212 with the mutation analysis mechanism. In general, the mutation analysis mechanism enables the genetic information processing system 100 to quickly and accurately determine whether an indel mutation exists in a sequence of the cancerous sample DNA information 112 of FIG. 1 that corresponds to a particular instance of the reference tandem repeat sequence 212.

It has been found that analysis of mutation patterns in the reference tandem repeat sequences 212 can be used to indicate the existence or possible development of a particular form of cancer. In particular, indel mutations have been found to occur at higher frequencies over substitution type mutations by an order of magnitude or more. Thus, using the reference tandem repeat sequence 212 to detect indel mutations with the tandem repeat indel variants 310 provides the benefit of being used as markers to detect development or existence of mutations that are linked to a particular form of cancer.

For the purposes of the mutation identification process, it is important that at least one of the tandem repeat indel variants 310 includes at least one instance of the associated tandem repeat indel k-mers 316 that does not exist within the reference genome due to the matching process used in the mutation analysis mechanism to identify corresponding sequences in the healthy sample DNA information 110 of FIG. 1 and the cancerous sample DNA information 112. For example, when one instance of the associated tandem repeat indel k-mers 316 for one of the tandem repeat indel variants 310 does not exist in the reference genome, a match between a sequence in the cancerous sample DNA information 112 and the specific instance of the associated tandem repeat indel k-mers 316 can verify that the particular indel mutation exists. However, the tandem repeat indel variants 310 that include more than one of the associated tandem repeat indel k-mers 316 that does not appear in the reference genome can prevent misidentification due to sequencing errors or point mutations in the flanking sequences. Thus, a minimum number of the tandem repeat indel variants 310 should not appear or exist in the reference genome in order to accurately identify when a sequence at a specific location includes an insertion mutation or a deletion mutation using the unique reference tandem repeats k-mer 210.

Instances of the unique reference tandem repeat k-mer 210 that can be used for the mutation identification process are referred to as indel analysis tandem repeat k-mers 314. The indel analysis tandem repeat k-mers 314 are a subset of the unique reference tandem repeat k-mer 210 with associated instances of the tandem repeat indel variants 310 that do not appear in the reference genome. In other words, the unique reference tandem repeat k-mer 210 is one of the indel analysis tandem repeat k-mers 314 if the reference tandem repeat sequence 212 included in the unique reference tandem repeat k-mer 210 also includes at least one of the tandem repeat indel variants 310 that does not appear in the reference genome. The genome tandem repeat reference catalogue 130 can identify which of the unique reference tandem repeat k-mer 210 for a particular instance of the reference tandem repeat sequence 212 is one of the indel analysis tandem repeat k-mers 314.

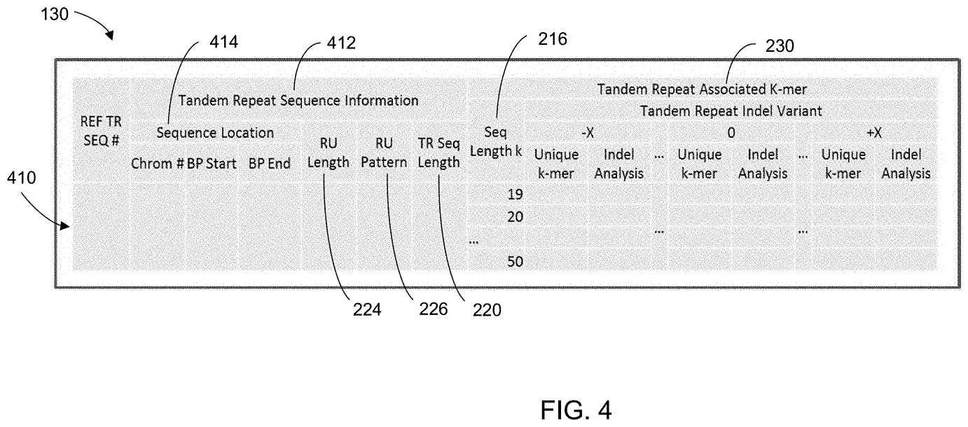

Referring now to FIG. 4, therein is shown an example illustration of an entry in the genome tandem repeat reference catalogue 130. The genome tandem repeat reference catalogue 130 can include catalogue entries 410 for each instance of the reference tandem repeat sequence 212. The catalogue entries 410 for each instance of the reference tandem repeat sequence 212 of FIG. 2 can include tandem repeat sequence information 412. The tandem repeat sequence information 412 is information that characterizes the reference tandem repeat sequence 212. For example, the tandem repeat sequence information 412 can include a sequence location 414, the tandem repeat sequence length 220, the repeat unit length 224 of the reference repeat unit 222, the repeat unit pattern 226 of the reference repeat unit 222, or a combination thereof.

The sequence location 414 is information about the location of the reference tandem repeat sequence 212 within the reference genome. As an example, the sequence location 414 can be described based on the molecular location of the tandem repeat sequence, which can include the chromosome on which the reference tandem repeat sequence 212 is located, and the base pair numbers in the chromosome that marks the beginning and end of the reference tandem repeat sequence 212. The sequence location 414 can act as a unique identifier that distinguishes one instance of the reference tandem repeat sequence 212 from one another. For example, multiple instances of the reference tandem repeat sequence 212 that share the same repeat unit pattern 226 and repeat unit length 224 can be distinguished from one another based on the sequence location 414 specific to each of the reference tandem repeat sequence 212.

The catalogue entries 410 for each instance of the reference tandem repeat sequence 212 can include information for one or more instances of the tandem repeat associated k-mers 230. For example, the catalogue entries 410 can include information for the tandem repeat associated k-mers 230 of various values of the sequence length k 216. For illustrative purposes, this instance of the catalogue entries 410 is shown including information for the tandem repeat associated k-mers 230 ranging from the sequence length k 216 of 19 base pairs to 50 base pairs, although it is understood that the catalogue entries 410 can include information about the tandem repeat associated k-mers 230 that are greater than 50 base pairs. As another example, the catalogue entries 410 can include information about which of the tandem repeat associated k-mers 230 that are the unique reference tandem repeat k-mers 210 of FIG. 2, the indel analysis tandem repeat k-mers 314 of FIG. 3, or a combination thereof. As a specific example, the catalogue entries 410 can include the total number and which of the tandem repeat associated k-mers 230 for a particular instance of the reference tandem repeat sequence 212 of the sequence length k 216 that are the unique reference tandem repeat k-mers 210. For instance an exact match analysis between the tandem repeat associated k-mers 316 all having the sequence length k 216 of 30 base pairs for the reference tandem repeat sequence 212 “A8” beginning at position 10,513,372 yields a total number of 16 sequences that are the unique reference tandem repeat k-mers 210.

As another specific example, the catalogue entries 410 can include the total number and which of tandem repeat indel variants 310 for a particular instance of the indel analysis tandem repeat k-mers 314 do not appear within the reference genome. For illustrative purposes, TABLE 1 below summarizes an exact match analysis between the associated tandem repeat indel k-mers 316 all having the sequence length k 216 of 30 base pairs for the reference tandem repeat sequence 212 “A8” beginning at position 10,513,372, annotated as '372, on chromosome 22. In this example, each of the associated tandem repeat indel k-mers 316 for each instance of the tandem repeat indel variant 310 with the indel variant value 312 ranging from “−5” to “5” do not appear in the reference genome, although this may not be the case for other instances of the reference tandem repeat sequence 212.

TABLE 1
Chromosome 22, ′372 “A8” Reference Tandem Repeat
Associated Tandem Repeat Indel K-mer Summary

| indel variant value | Position Variant Total | Total that do not appear |
|---|---|---|
| 5 | 16 | 16 |
| 4 | 17 | 17 |
| 3 | 18 | 18 |
| 2 | 19 | 19 |
| 1 | 20 | 20 |
| −1 | 22 | 22 |
| −2 | 23 | 23 |
| −3 | 24 | 24 |
| −4 | 25 | 25 |
| −5 | 26 | 26 |

The genome tandem repeat reference catalogue 130 illustrated in FIG. 4 is shown for exemplary purposes as a template with a general layout for organizing information for each of the reference tandem repeat sequences 212. It is understood that the information for the reference tandem repeat sequences 212, including the tandem repeat sequence information 412, can include different categorizations and arrangements with additional or different pieces of information. Further, it is understood that an active or in-use version of the genome tandem repeat reference catalogue 130 will be populated with values corresponding to the various categories of the catalogue entries 410.

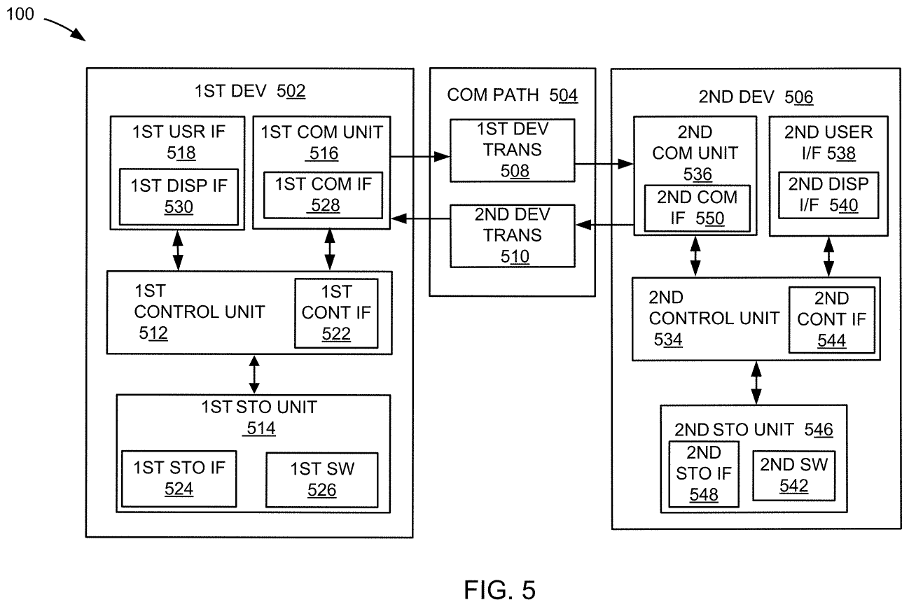

Referring now to FIG. 5, therein is shown an exemplary block diagram of the genetic information processing system 100. The genetic information processing system 100 can be implemented on a first device 502, a second device 506, or a combination thereof. The first device 502 can be the computing device 102 of FIG. 1. The first device 502 can couple, either directly or indirectly, to the communication path 504 to communicate with the second device 506 or can be a stand-alone device.

The second device 506 can be any of a variety of centralized or decentralized computing devices. For example, the second device 506 can be a multimedia computer, a laptop computer, a desktop computer, grid-computing resources, a virtualized computer resource, cloud computing resource, routers, switches, peer-to-peer distributed computing devices, DNA sequencing device, or a combination thereof.

The second device 506 can be centralized in a single room, distributed across different rooms, distributed across different geographical locations, embedded within a telecommunications network. The second device 506 can couple with the communication path 504 to communicate with the first device 502.

For illustrative purposes, the genetic information processing system 100 is described with the first device 502 as a computing device 102, although it is understood that the second device 506 can be the computing device 102. Also for illustrative purposes, the computing system 200 is shown with the second device 506 and the first device 502 as end points of the communication path 504, although it is understood that the genetic information processing system 100 can have a different partition between the first device 502, the second device 506, and the communication path 504. For example, the first device 502, the second device 506, or a combination thereof can also function as part of the communication path 504.

The communication path 504 can span and represent a variety of networks and network topologies. For example, the communication path 504 can include wireless communication, wired communication, optical, ultrasonic, or the combination thereof. Satellite communication, cellular communication, Bluetooth, Infrared Data Association standard (lrDA), wireless fidelity (WiFi), and worldwide interoperability for microwave access (WiMAX) are examples of wireless communication that can be included in the communication path 504. Ethernet, digital subscriber line (DSL), fiber to the home (FTTH), and plain old telephone service (POTS) are examples of wired communication that can be included in the communication path 504. Further, the communication path 504 can traverse a number of network topologies and distances. For example, the communication path 504 can include direct connection, personal area network (PAN), local area network (LAN), metropolitan area network (MAN), wide area network (WAN), or a combination thereof.

The first device 502 can send information in a first device transmission 508 over the communication path 504 to the second device 506. The second device 506 can send information in a second device transmission 510 over the communication path 504 to the first device 502.

The first device 502 can include a first control unit 512, a first storage unit 514, a first communication unit 516, and a first user interface 518. The first control unit 512 can include a first control interface 522. The first control unit 512 can execute a first software 526 to provide the intelligence of the computing system 200.

The first control unit 512 can be implemented in a number of different manners. For example, the first control unit 512 can be a processor, an application specific integrated circuit (ASIC) an embedded processor, a microprocessor, a hardware control logic, a hardware finite state machine (FSM), a digital signal processor (DSP), or a combination thereof. The first control interface 522 can be used for communication between the first control unit 512 and other functional units in the first device 502. The first control interface 522 can also be used for communication that is external to the first device 502.

The first control interface 522 can receive information from the other functional units or from external sources, or can transmit information to the other functional units or to external destinations. The external sources and the external destinations refer to sources and destinations external to the first device 502.

The first control interface 522 can be implemented in different ways and can include different implementations depending on which functional units or external units are being interfaced with the first control interface 522. For example, the first control interface 522 can be implemented with a pressure sensor, an inertial sensor, a microelectromechanical system (MEMS), optical circuitry, waveguides, wireless circuitry, wireline circuitry, or a combination thereof.

The first storage unit 514 can store the first software 526. The first storage unit 514 can also store the relevant information. For example, first storage unit 514 can include the genome tandem repeat reference catalogue 130 of FIG. 1 the DNA sample set 106 of FIG. 1, or a combination thereof.

The first storage unit 514 can be a volatile memory, a nonvolatile memory, an internal memory, an external memory, or a combination thereof. For example, the first storage unit 514 can be a nonvolatile storage such as non-volatile random access memory (NVRAM), Flash memory, disk storage, or a volatile storage such as static random access memory (SRAM).

The first storage unit 514 can include a first storage interface 524. The first storage interface 524 can be used for communication between and other functional units in the first device 502. The first storage interface 524 can also be used for communication that is external to the first device 502.

The first storage interface 524 can receive information from the other functional units or from external sources, or can transmit information to the other functional units or to external destinations. The external sources and the external destinations refer to sources and destinations external to the first device 502.

The first storage interface 524 can include different implementations depending on which functional units or external units are being interfaced with the first storage unit 514. The first storage interface 524 can be implemented with technologies and techniques similar to the implementation of the first control interface 522.

The first communication unit 516 can enable external communication to and from the first device 502. For example, the first communication unit 516 can permit the first device 502 to communicate with the second device 506 of FIG. 1, an attachment, such as a peripheral device or a computer desktop, and the communication path 504.

The first communication unit 516 can also function as a communication hub allowing the first device 502 to function as part of the communication path 504 and not limited to be an end point or terminal unit to the communication path 504. The first communication unit 516 can include active and passive components, such as microelectronics or an antenna, for interaction with the communication path 504.

The first communication unit 516 can include a first communication interface 528. The first communication interface 528 can be used for communication between the first communication unit 516 and other functional units in the first device 502. The first communication interface 528 can receive information from the other functional units or can transmit information to the other functional units.

The first communication interface 528 can include different implementations depending on which functional units are being interfaced with the first communication unit 516. The first communication interface 528 can be implemented with technologies and techniques similar to the implementation of the first control interface 522.

The first user interface 518 allows a user (not shown) to interface and interact with the first device 502. The first user interface 518 can include an input device and an output device. Examples of the input device of the first user interface 518 can include a keypad, a touchpad, soft-keys, a keyboard, a microphone, an infrared sensor for receiving remote signals, or any combination thereof to provide data and communication inputs.

The first user interface 518 can include a first display interface 530. The first display interface 530 can include a display, a projector, a video screen, a speaker, or any combination thereof.

The first control unit 512 can operate the first user interface 518 to display information generated by the computing system 200. The first control unit 512 can also execute the first software 526 for the other functions of the computing system 200. The first control unit 512 can further execute the first software 526 for interaction with the communication path 504 via the first communication unit 516.

The second device 506 can be optimized for implementing an embodiment of the present invention in a multiple device embodiment with the first device 502. The second device 506 can provide the additional or higher performance processing power compared to the first device 502. The second device 506 can include a second control unit 534, a second communication unit 536, and a second user interface 538.

The second user interface 538 allows a user (not shown) to interface and interact with the second device 506. The second user interface 538 can include an input device and an output device. Examples of the input device of the second user interface 538 can include a keypad, a touchpad, soft-keys, a keyboard, a microphone, or any combination thereof to provide data and communication inputs. Examples of the output device of the second user interface 538 can include a second display interface 540. The second display interface 540 can include a display, a projector, a video screen, a speaker, or any combination thereof.

The second control unit 534 can execute a second software 542 to provide the intelligence of the second device 506 of the computing system 200. The second software 542 can operate in conjunction with the first software 526. The second control unit 534 can provide additional performance compared to the first control unit 512.

The second control unit 534 can operate the second user interface 538 to display information. The second control unit 534 can also execute the second software 542 for the other functions of the computing system 200, including operating the second communication unit 536 to communicate with the first device 502 over the communication path 504.

The second control unit 534 can be implemented in a number of different manners. For example, the second control unit 534 can be a processor, an embedded processor, a microprocessor, hardware control logic, a hardware finite state machine (FSM), a digital signal processor (DSP), or a combination thereof.

The second control unit 534 can include a second controller interface 544. The second controller interface 544 can be used for communication between the second control unit 534 and other functional units in the second device 506. The second controller interface 544 can also be used for communication that is external to the second device 506.

The second controller interface 544 can receive information from the other functional units or from external sources, or can transmit information to the other functional units or to external destinations. The external sources and the external destinations refer to sources and destinations external to the second device 506.

The second controller interface 544 can be implemented in different ways and can include different implementations depending on which functional units or external units are being interfaced with the second controller interface 544. For example, the second controller interface 544 can be implemented with a pressure sensor, an inertial sensor, a microelectromechanical system (MEMS), optical circuitry, waveguides, wireless circuitry, wireline circuitry, or a combination thereof.

A second storage unit 546 can store the second software 542. The second storage unit 546 can also store the genome tandem repeat reference catalogue 130 of FIG. 1, the DNA sample set 106 of FIG. 1, or a combination thereof. The second storage unit 546 can be sized to provide the additional storage capacity to supplement the first storage unit 514.

For illustrative purposes, the second storage unit 546 is shown as a single element, although it is understood that the second storage unit 546 can be a distribution of storage elements. Also for illustrative purposes, the computing system 200 is shown with the second storage unit 546 as a single hierarchy storage system, although it is understood that the computing system 200 can have the second storage unit 546 in a different configuration. For example, the second storage unit 546 can be formed with different storage technologies forming a memory hierarchal system including different levels of caching, main memory, rotating media, or off-line storage.

The second storage unit 546 can be a volatile memory, a nonvolatile memory, an internal memory, an external memory, or a combination thereof. For example, the second storage unit 546 can be a nonvolatile storage such as non-volatile random access memory (NVRAM), Flash memory, disk storage, or a volatile storage such as static random access memory (SRAM).

The second storage unit 546 can include a second storage interface 548. The second storage interface 548 can be used for communication between other functional units in the second device 506. The second storage interface 548 can also be used for communication that is external to the second device 506.

The second storage interface 548 can receive information from the other functional units or from external sources, or can transmit information to the other functional units or to external destinations. The external sources and the external destinations refer to sources and destinations external to the second device 506.

The second storage interface 548 can include different implementations depending on which functional units or external units are being interfaced with the second storage unit 546. The second storage interface 548 can be implemented with technologies and techniques similar to the implementation of the second controller interface 544.

The second communication unit 536 can enable external communication to and from the second device 506. For example, the second communication unit 536 can permit the second device 506 to communicate with the first device 502 over the communication path 504.

The second communication unit 536 can also function as a communication hub allowing the second device 506 to function as part of the communication path 504 and not limited to be an end point or terminal unit to the communication path 504. The second communication unit 536 can include active and passive components, such as microelectronics or an antenna, for interaction with the communication path 504.

The second communication unit 536 can include a second communication interface 550. The second communication interface 550 can be used for communication between the second communication unit 536 and other functional units in the second device 506. The second communication interface 550 can receive information from the other functional units or can transmit information to the other functional units.

The second communication interface 550 can include different implementations depending on which functional units are being interfaced with the second communication unit 536. The second communication interface 550 can be implemented with technologies and techniques similar to the implementation of the second controller interface 544.

The first communication unit 516 can couple with the communication path 504 to send information to the second device 506 in the first device transmission 508. The second device 506 can receive information in the second communication unit 536 from the first device transmission 508 of the communication path 504.

The second communication unit 536 can couple with the communication path 504 to send information to the first device 502 in the second device transmission 510. The first device 502 can receive information in the first communication unit 516 from the second device transmission 510 of the communication path 504. The computing system 200 can be executed by the first control unit 512, the second control unit 534, or a combination thereof. For illustrative purposes, the second device 506 is shown with the partition having the second user interface 538, the second storage unit 546, the second control unit 534, and the second communication unit 536, although it is understood that the second device 506 can have a different partition. For example, the second software 542 can be partitioned differently such that some or all of its function can be in the second control unit 534 and the second communication unit 536. Also, the second device 506 can include other functional units not shown in FIG. 5 for clarity.

The functional units in the first device 502 can work individually and independently of the other functional units. The first device 502 can work individually and independently from the second device 506 and the communication path 504.

The functional units in the second device 506 can work individually and independently of the other functional units. The second device 506 can work individually and independently from the first device 502 and the communication path 504.

For illustrative purposes, the genetic information analysis system 100 is described by operation of the first device 502 and the second device 506. It is understood that the first device 502 and the second device 506 can operate any of the modules and functions of the genetic information analysis system 100.

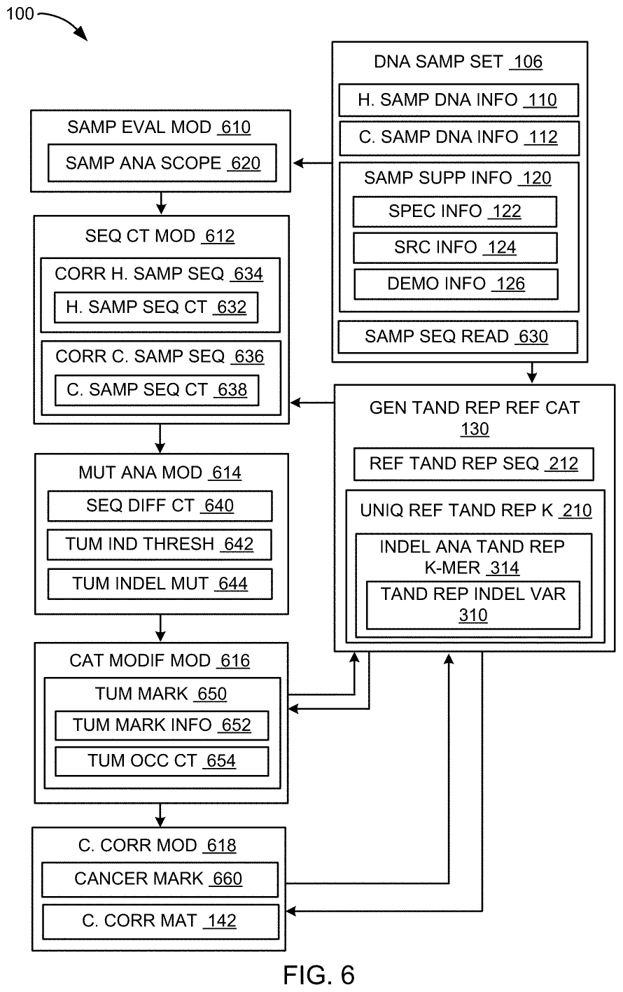

Referring now to FIG. 6, therein is shown a control flow for the functions of the genetic information analysis system 100. The genetic information analysis system 100 can be implemented to supplement and refine information in the genome tandem repeat reference catalogue 130 with information from the DNA sample sets 106 based on the reference tandem repeat sequences 212. In general, the genetic information processing system 100 can analyze one or more of the DNA sample sets 106 to determine the existence of mutations in specific locations of DNA sequences, correlation of mutation patterns to determine indications of cancer, or a combination thereof. The functions of the genetic information processing system 100 can be implemented with a sample set evaluation module 610, a sequence count module 612, a mutation analysis module 614, a catalogue modification module 616, a cancer correlation module 618, or a combination thereof. The sequence count module 612 can be coupled to the sample set evaluation module 610. The mutation analysis module 614 can be coupled to the sequence count module 612. The catalogue modification module 616 can be coupled to the mutation analysis module 614. The cancer correlation module 618 can be coupled to the mutation analysis module 614, the catalogue modification module 616, or a combination thereof.

The genetic information processing system 100 can evaluate the scope of the DNA sample set 106, including the healthy sample DNA information 110 and the cancerous sample DNA information 112, with the sample set evaluation module 610. For example, the sample set evaluation module 610 can evaluate the DNA sample set 106 to identify factors and properties of the DNA sample set 106 to facilitate analysis of the healthy sample DNA information 110 and the cancerous sample DNA information 112 with the mutation analysis mechanism. The implementation of the sample set evaluation module 610 can be optional. The sample set evaluation module 610 can generate a sample analysis scope 620 for the DNA sample set 106. The sample analysis scope 620 is a set of one or more factors to determine how the DNA sample set 106 is analyzed. For example, the sample analysis scope 620 can be based on the sample supplemental information 120 of the DNA sample set 106, such as the sample specification information 122, to identify the indel analysis tandem repeat k-mers 314 that can be used based on sequence location 414 and sequence length k 216 of the sequences in the healthy sample DNA information 110, the cancerous sample DNA information 112, or a combination thereof.

The genetic information processing system 100 can, in one implementation, receive the indel analysis tandem repeat k-mer 314 and associated information from the genome tandem repeat reference catalogue 130, the DNA sample set 106, or a combination thereof for processing by the mutation analysis mechanism. The mutation analysis mechanism of the genetic information processing system 100 can be implemented with the sequence count module 612 and the mutation analysis module 614. The sequence count module 612 is for calculating a sequence count for specific DNA sequences in a sample set that corresponds to a reference sequence. The sequence count module 612 can calculate the sequence count based on the number of sample sequence reads 630, which are the sequence reads for the DNA fragments for the healthy sample DNA information 110, the cancerous sample DNA information 112, or a combination thereof.

For the healthy sample DNA information 110, the sequence count module 612 can calculate a healthy sample sequence count 632 for each instance of a corresponding healthy sample sequence 634 identified in the healthy sample DNA information 110. The corresponding healthy sample sequence 634 is a DNA sequence in the healthy sample DNA information 110 that corresponds to one of the tandem repeat indel variants 310 for a particular one of the indel analysis tandem repeat k-mers 314. The healthy sample sequence count 632 is the number of times the corresponding healthy sample sequence 634 is identified in the healthy sample DNA information set 110.

Similarly, for the cancerous sample DNA information 112, the sequence count module 612 can calculate a cancerous sample sequence count 636 for each instance of a corresponding cancerous sample sequence 638 identified in the cancerous sample DNA information 112. The corresponding cancerous sample sequence 638 is a DNA sequence in the cancerous sample DNA information 112 that corresponds to one of the tandem repeat indel variants 310 for a particular one of the indel analysis tandem repeat k-mers 314. The cancerous sample sequence count 636 is the number of times the corresponding cancerous sample sequence 638 is identified in the cancerous sample DNA information set 112.

The sequence count module 612 can identify the corresponding healthy sample sequence 634 and the corresponding cancerous sample sequence 638 for a given instance of the unique reference tandem repeat k-mer 210, and more specifically the indel analysis tandem repeat k-mers 314. For example, the sequence count module 612 can search through the healthy sample DNA information 110 of the DNA sample set 106 and the cancerous sample DNA information 112, respectively, for matches to one or more of the tandem repeat indel variants 310 of the indel analysis tandem repeat k-mers 314. As one specific example, the sequence count module 612 can search for a string of consecutive base pairs that exactly matches with one of the tandem repeat indel variants 310 of the indel analysis tandem repeat k-mers 314.

The sequence count module 612 can calculate the healthy sample sequence count 632 as the total number of each of the corresponding healthy sample sequence 634 identified in each of the sample sequence reads 630 in the healthy sample DNA information 110. In many cases, the corresponding healthy sample sequence 634 will correspond with a single instance of the tandem repeat indel variants 310. In these cases, the total value of the healthy sample sequence count 632 will be equal to the total number of the sample sequence reads 630 in the healthy sample DNA information set 110. For example, where the healthy sample DNA information set 110 includes 50 instances of the sample sequence reads 630 per DNA segment, the healthy sample sequence count 632 for a given instance of the corresponding healthy sample sequence 634 should also be 50. The case of non-unity between the number of sequence reads and the healthy sample sequence count 632 can generally be attributed to sequencing errors.

In many cases, the corresponding healthy sample sequence 634 will match with the indel analysis tandem repeat k-mer 314 with the indel variant value 312 zero, which is the unique reference tandem repeat k-mer 210 including the reference tandem repeat sequence 212 having no insertions or deletions of the reference repeat unit 222. However, in some cases, the corresponding healthy sample sequence 634 can differ. The differences between the corresponding healthy sample sequence 634 and the indel analysis tandem repeat k-mers 314 with the indel variant value 312 zero can account for wild type variations, or naturally occurring variations, in the healthy sample DNA information 110.

Similarly, the sequence count module 612 can calculate the cancerous sample sequence count 636 for each of the corresponding cancerous sample sequence 638 that appear in the sample sequence reads 630 in the cancerous sample DNA information 112. Due to possible mutations, the cancerous sample DNA information 112 can include multiple different instances of the corresponding cancerous sample sequence 638 matching to different instances of the tandem repeat indel variants 310, with each corresponding cancerous sample sequence 638 having varying values of the cancerous sample sequence count 636. As an example, in some cases, the corresponding cancerous sample sequence 638 and cancerous sample sequence count 636 will match with the corresponding healthy sample sequence 634 and healthy sample sequence count 632, indicating no mutations. As another example, for a given instance of the indel analysis tandem repeat k-mers 314, the cancerous sample DNA information 112 will have a split in the cancerous sample sequence count 636 between the corresponding cancerous sample sequence 638 that is the same as the corresponding healthy sample sequence 634 and one or more other instances of the tandem repeat indel variants 310. For a given instance of the indel analysis tandem repeat k-mers 314, the sequence count module 612 can track the cancerous sample sequence count 636 for each different instance of the corresponding cancerous sample sequence 638 in the cancerous sample DNA information 112.

The flow can continue to the mutation analysis module 614. The mutation analysis module 614 is for determining whether a mutation exists in the corresponding cancerous sample sequence 638 of the cancerous sample DNA information 112. In general, the existence of a mutation in the cancerous sample DNA information 112 can be determined based on differences in the reference tandem repeat sequence 212 between the corresponding healthy sample sequence 634 and the corresponding cancerous sample sequence 638. More specifically, difference in the number of the reference repeat unit 222 can represent the existence of an indel mutation, which is the mutation due to an insertion or deletion of the reference repeat unit 222 in the corresponding cancerous sample sequence 638 relative to the corresponding healthy sample sequence 634. For example, the mutation analysis module 614 can determine that a mutation exists when the corresponding cancerous sample sequence 638 matches one of the tandem repeat indel variant 310 that is different from that of the corresponding healthy sample sequence 634. In another example, the mutation analysis module 614 can determine the difference between the corresponding healthy sample sequence 634 and the corresponding cancerous sample sequence 638 based on a sequence difference count 640. The sequence difference count 640 is the total number of corresponding cancerous sample sequence 638 that differ from the corresponding healthy sample sequence 634. In the case where the sequence difference count 640 indicates no differences, such as when the sequence difference count 640 is zero, the mutation analysis module 614 can determine that no mutation exists in the corresponding cancerous sample sequence 638.

In general, the mutation analysis module 614 can determine that the indel mutation has occurred when the sequence difference count 640 is a non-zero value. For example, in one implementation, the mutation analysis module 614 can determine whether the indel mutation is the tumorous indel mutation when the sequence difference count 640 is greater than the sequencing error percentage for the methods used to sequence the healthy sample DNA information 110, the cancerous sample DNA information 112, or a combination thereof.

In another implementation, mutation analysis module 614 can determine whether the indel mutation is a tumorous indel mutation 644 based on a tumor indication threshold 642. The tumor indication threshold 642 is an indicator of whether the number of mutations for a particular sequence in the cancerous sample DNA information 112 indicates the existence of a tumorous indel mutation 644. The tumorous indel mutation 644 occurs when the sequence difference count 640 exceeds the tumor indication threshold 642. As an example, the tumor indication threshold 642 can be based on a percentage between the total number of the sample sequence reads 630 and the sequence difference count 640. As a specific example, the tumor indication threshold 642 can be when the sequence difference count 640 greater than 70% of the sample sequence reads 630 for the cancerous sample DNA information 112. In another specific example, the tumor indication threshold 642 can be when the sequence difference count 640 is greater than 80% of the sample sequence reads 630 for the cancerous sample DNA information 112. In a further specific example, the tumor indication threshold 642 can be when the sequence difference count 640 greater than 90% of the sample sequence reads 630 for the cancerous sample DNA information 112.

When the corresponding cancerous sample sequence 638 includes the tumorous indel mutation 644, the genetic information processing system 100 can implement the catalogue modification module 616 to update or modify the genome tandem repeat reference catalogue 130. For example, the catalogue modification module 616 can modify the genome tandem repeat reference catalogue 130 by identifying the instance of the catalogue entries 410 for the reference tandem repeat sequence 212 as a tumor marker 650 when the tumorous indel mutation 644 exists in the corresponding cancerous sample sequence 638.

The catalogue entries 410 of FIG. 4 for the reference tandem repeat sequences 212 identified as the tumor marker 650 can be modified by the catalogue modification module 616 to include tumor marker information 652. The tumor marker information 652 is information characterizing the tumor. For example, the tumor marker information 652 can include a tumor occurrence count 654, which is a count of the number of times the tumorous indel mutation 644 was identified in a particular instance of the reference tandem repeat sequence 212 for a given form of cancer. As a specific example the tumor occurrence count 654 can be compiled from analysis of the DNA sample set 106 for numerous cancer patients.

In another example, the tumor marker information 652 can include information about the different instances of the corresponding cancerous sample sequence 638 matching to different instances of tandem repeat indel variants 310 along with the cancerous sample sequence count 636, the total number of the sample sequence reads 630 of the DNA sample set 106, all or portions of the sample supplemental information 120 for the DNA sample set 106, or a combination thereof. In a further example, the tumor marker information 652 can include the number of the reference repeat unit 222 in the corresponding cancerous sample sequence 638 that were different form the corresponding healthy sample sequence 634.

The tumor marker information 652 can include information based on the sample supplemental information 120. For example, the tumor marker information 652 can include the sample supplemental information 120 of the sample source information 124, such as the cancer type, the stage of cancer development, organ or tissue form which the sample was extracted, or a combination thereof. In another example, the tumor marker information 652 can include the sample supplemental information 120 of the patient demographic information 126, such as the age, the gender, the ethnicity, geographic location of where the patient resides or has been, the duration of time the patient stayed or resided at the geographic location, predispositions for genetic disorders or cancer development, or a combination thereof.

The genetic information processing system 100 can use one or more instances of the reference tandem repeat sequence 212 identified as the tumor marker 650 to generate the cancer correlation matrix 142 with the cancer correlation module 618. For example, the cancer correlation module 618 can identify cancer markers 660 based on the tumor occurrence count 654 for each of the tumor markers 650 in the genome tandem repeat reference catalogue 130. The cancer markers 660 are mutation hotspots specific to indel mutations in instances of the reference tandem repeat sequence 212. In one implementation, the cancer correlation module 618 can identify the cancer markers 660 based on regression analysis. For example, the regression analysis can be performed with a receiver operating characteristic curve to the optimum sensitivity and specificity from the tumor markers 650, tumor occurrence count 654, or a combination thereof to determine the cancer markers 660.

In another implementation, the cancer correlation module 618 can identify the cancer markers 660 based on a ratio between or percentage of the tumor occurrence count 654 for the tumor marker 650 and the total number of the DNA sample sets 106 of a particular form of cancer that have been analyzed for the tumor marker 650. As a specific example, the cancer correlation module 618 can identify the cancer markers 660 as the tumor markers 650 when the ratio between the tumor occurrence count 654 and the total number of the DNA sample sets 106 analyzed is 90% or more of the DNA sample sets 106 analyzed for a particular form of cancer. In this case, the cancer correlation matrix 142 can include the cancer markers 660 that were identified in this manner.

In a further implementation, the cancer correlation module 618 generate the cancer correlation matrix 142 as the tumor markers 650 that are common among a percentage of the DNA sample sets 106 for a particular form of cancer. For example, the cancer correlation module 618 can generate the cancer correlation matrix 142 as the tumor markers 650 that appear in 90% or more of the total number of the DNA sample sets 106. In other implementations, the cancer correlation module 618 can generate the cancer correlation matrix 142 through other methods, such as regression analysis, or clustering.

The cancer correlation module 618 can generate the cancer correlation matrix 142 taking into account the sample supplemental information 120, such as the patient demographic information 126, to generate the cancer correlation matrix 142 for sub-populations. For example, the cancer correlation module 618 can generate the cancer correlation matrix 142 based on the patient demographic information 126 specific to gender, nationality, geographic location, occupation, age, or other characteristic.

The genetic information processing system 100 has been described with module functions or order as an example. The genetic information processing system 100 can partition the modules differently or order the modules differently. For example, the sample set evaluation module 610 can be implemented on the second device 506 and the sequence count module 612, the mutation analysis module 614 and the cancer correlation module 618 can be implemented on the first device 502.

For illustrative purposes, the various modules have been described as being specific to the first device 502 or the second device 506. However, it is understood that the modules can be distributed differently. For example, the various modules can be implemented in a different device, or the functionalities of the modules can be distributed across multiple devices. Also as an example, the various modules can be stored in a non-transitory memory medium.

As a more specific example, one or more modules described above can be stored in the non-transitory memory medium for distribution to a different system, a different device, a different user, or a combination thereof, for manufacturing, or a combination thereof. Also as a more specific example, the modules described above can be implemented or stored using a single hardware unit, such as a chip or a processor, or across multiple hardware units.

The modules described in this application can be hardware implementation or hardware accelerators in the first control unit 516 of FIG. 5 or in the second control unit 538 of FIG. 5. The modules can also be hardware implementation or hardware accelerators within the first device 502 or the second device 506 but outside of the first control unit 516 or the second control unit 538, respectively, as depicted in FIG. 5. However, it is understood that the first control unit 516, the second control unit 538, or a combination thereof can collectively refer to all hardware accelerators for the modules.

The modules described in this application can be implemented as instructions stored on a non-transitory computer readable medium to be executed by the first control unit 512, the second control unit 536, or a combination thereof. The non-transitory computer medium can include the first storage unit 514 of FIG. 5, the second storage unit 546 of FIG. 5, or a combination thereof. The non-transitory computer readable medium can include non-volatile memory, such as a hard disk drive, non-volatile random access memory (NVRAM), solid-state storage device (SSD), compact disk (CD), digital video disk (DVD), or universal serial bus (USB) flash memory devices. The non-transitory computer readable medium can be integrated as a part of the genetic information processing system 100 or installed as a removable portion of the genetic information processing system 100.

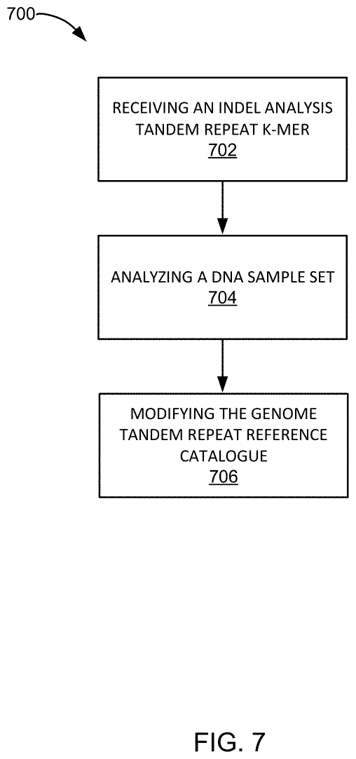

Referring now to FIG. 7, therein is shown a flow chart of a method 700 of operation of the genetic information processing system 100 in an embodiment of the present invention.

The method 700 includes: receiving an indel analysis tandem repeat k-mer of sequence length-k nucleotides a genome tandem repeat reference catalogue, wherein the indel analysis tandem repeat k-mer is unique within a reference human genome and include: a reference tandem repeat sequence; and flanking sequences directly preceding and following the reference tandem repeat sequence in a block 702; analyzing a DNA sample set, including a healthy sample DNA information and a cancerous sample DNA information, based on the genome tandem repeat reference catalogue including: identify a corresponding healthy sample sequence in the healthy sample DNA information and a corresponding cancerous sample sequence in the cancerous sample DNA information corresponding the indel analysis tandem repeat k-mer; determine whether an indel mutation exists in a corresponding tandem repeat sequence of the corresponding cancerous sample sequence based on a comparison to the corresponding healthy sample sequence in a block 704; and modifying the genome tandem repeat reference catalogue to identify the reference tandem repeat sequence of the instance of indel analysis tandem repeat k-mer as a tumor marker when the tumorous indel mutation exists in the corresponding cancerous sample sequence in a block 706.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization. Another important aspect of an embodiment of the present invention is that it valuably supports and services the historical trend of reducing costs, simplifying systems, and increasing performance.

These and other valuable aspects of an embodiment of the present invention consequently further the state of the technology to at least the next level. While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters set forth herein or shown in the accompanying drawings are to be interpreted in an illustrative and non-limiting sense.

What is claimed is:

1. A genetic information processing system comprising:
a control unit configured to:
receive an indel analysis tandem repeat k-mer of sequence length k nucleotides from a genome tandem repeat reference catalogue, wherein the indel analysis tandem repeat k-mer is unique within a reference human genome and include:
a reference tandem repeat sequence; and
flanking sequences directly preceding and following the reference tandem repeat sequence;
analyze a DNA sample set, including a healthy sample DNA information and a cancerous sample DNA information, based on the genome tandem repeat reference catalogue including:
identify a corresponding healthy sample sequence in the healthy sample DNA information and a corresponding cancerous sample sequence in the cancerous sample DNA information corresponding the indel analysis tandem repeat k-mer;
determine whether the corresponding cancerous sample sequence includes a tumorous indel mutation based on a comparison between the corresponding cancerous sample sequence and the corresponding healthy sample sequence; and
modify the genome tandem repeat reference catalogue to identify the reference tandem repeat sequence of the instance of indel analysis tandem repeat k-mer as a tumor marker when the tumorous indel mutation exists in the corresponding cancerous sample sequence.

2. The system of claim 1, wherein the control unit is configured to generate a cancer correlation matrix based on the tumor marker.

3. The system of claim 1, wherein the control unit is configured to identify the corresponding healthy sample sequence as a wild type sequence based on differences between the reference tandem repeat sequence and the corresponding healthy sample sequence.

4. The system of claim 1, wherein the control unit is configured to modify the genome tandem repeat reference catalogue with a sample supplemental information of the cancerous sample DNA information for the reference tandem repeat sequence identified as the tumor marker.

5. The system of claim 1, wherein the sequence length k of the indel analysis tandem repeat k-mer is a minimum of 19 nucleotide base pairs.

6. The system of claim 1, wherein the reference tandem repeat sequence includes a tandem repeat sequence length of at least five nucleotide base pairs.

7. The system of claim 1, wherein the reference tandem repeat sequence includes a reference repeat unit with a tandem repeat pattern of a mono-nucleotide pattern, a di-nucleotide pattern, a tri-nucleotide pattern, or a tetra-nucleotide pattern.

8. A method of operation of the genetic information processing system comprising:
receiving an indel analysis tandem repeat k-mer of sequence length k nucleotides a genome tandem repeat reference catalogue, wherein the indel analysis tandem repeat k-mer is unique within a reference human genome and include:
a reference tandem repeat sequence; and
flanking sequences directly preceding and following the reference tandem repeat sequence;
analyzing a DNA sample set, including a healthy sample DNA information and a cancerous sample DNA information, based on the genome tandem repeat reference catalogue including:
identify a corresponding healthy sample sequence in the healthy sample DNA information and a corresponding cancerous sample sequence in the cancerous sample DNA information corresponding the indel analysis tandem repeat k-mer;
determine whether the corresponding cancerous sample sequence includes a tumorous indel mutation based on a comparison between the corresponding cancerous sample sequence and the corresponding healthy sample sequence; and
modifying the genome tandem repeat reference catalogue to identify the reference tandem repeat sequence of the instance of indel analysis tandem repeat k-mer as a tumor marker when the tumorous indel mutation exists in the corresponding cancerous sample sequence.

9. The method of claim 8, further comprising generating a cancer correlation matrix based on the tumor marker.

10. The method of claim 8, further comprising identifying the corresponding healthy sample sequence as a wild type sequence based on differences between the reference tandem repeat sequence and the corresponding healthy sample sequence.

11. The method of claim 8, wherein modifying the genome tandem repeat reference catalogue includes modifying the genome tandem repeat reference catalogue with a sample supplemental information of the cancerous sample DNA information for the reference tandem repeat sequence identified as the tumor marker.

12. The method of claim 8, wherein the sequence length k of the indel analysis tandem repeat k-mer is a minimum of 19 nucleotide base pairs.

13. The method of claim 8, wherein the reference tandem repeat sequence includes a tandem repeat sequence length of at least five nucleotide base pairs.

14. The method of claim 8, wherein the reference tandem repeat sequence includes a reference repeat unit with a tandem repeat pattern of a mono-nucleotide pattern, a di-nucleotide pattern, a tri-nucleotide pattern, or a tetra-nucleotide pattern.

15. A non-transitory computer readable medium including instructions executable by a control circuit for a genetic information processing system, the instructions comprising:
receiving an indel analysis tandem repeat k-mer of sequence length k nucleotides a genome tandem repeat reference catalogue, wherein the indel analysis tandem repeat k-mer is unique within a reference human genome and include:
a reference tandem repeat sequence; and
flanking sequences directly preceding and following the reference tandem repeat sequence;
analyzing a DNA sample set, including a healthy sample DNA information and a cancerous sample DNA information, based on the genome tandem repeat reference catalogue including:
identify a corresponding healthy sample sequence in the healthy sample DNA information and a corresponding cancerous sample sequence in the cancerous sample DNA information corresponding the indel analysis tandem repeat k-mer;
determine whether the corresponding cancerous sample sequence includes a tumorous indel mutation based on a comparison between the corresponding cancerous sample sequence and the corresponding healthy sample sequence; and
modifying the genome tandem repeat reference catalogue to identify the reference tandem repeat sequence of the instance of indel analysis tandem repeat k-mer as a tumor marker when the tumorous indel mutation exists in the corresponding cancerous sample sequence.

16. The non-transitory computer readable medium as claimed in claim 15 further comprising generating a cancer correlation matrix based on the tumor marker.

17. The non-transitory computer readable medium as claimed in claim 15 further comprising identifying the corresponding healthy sample sequence as a wild type sequence based on differences between the reference tandem repeat sequence and the corresponding healthy sample sequence.

18. The non-transitory computer readable medium as claimed in claim 15 wherein modifying the genome tandem repeat reference catalogue includes modifying the genome tandem repeat reference catalogue with a sample supplemental information of the cancerous sample DNA information for the reference tandem repeat sequence identified as the tumor marker.

19. The non-transitory computer readable medium as claimed in claim 15 wherein the sequence length k of the indel analysis tandem repeat k-mer is a minimum of 19 nucleotide base pairs.

20. The non-transitory computer readable medium as claimed in claim 15 wherein the reference tandem repeat sequence includes a tandem repeat sequence length of at least five nucleotide base pairs.


SEQUENCE LISTING (8 sequences)

SEQ ID NO: 1 - DNA, 34 bp, Homo sapiens
ttggataacctagaaaaaaaacaaattactggaa

SEQ ID NO: 2 - DNA, 30 bp, Homo sapiens
ggataacctagaaaaaaaaaaacaaattac

SEQ ID NO: 3 - DNA, 30 bp, Homo sapiens
ggataacctagaaaaaaaaaacaaattact

SEQ ID NO: 4 - DNA, 30 bp, Homo sapiens
ggataacctagaaaaaaaaacaaattactg

SEQ ID NO: 5 - DNA, 30 bp, Homo sapiens
ggataacctagaaaaaaaacaaattactgg

SEQ ID NO: 6 - DNA, 30 bp, Homo sapiens
ggataacctagaaaaaaacaaattactgga

SEQ ID NO: 7 - DNA, 30 bp, Homo sapiens
ggataacctagaaaaaacaaattactggaa

SEQ ID NO: 8 - DNA, 30 bp, Homo sapiens
ggataacctagaaaaacaaattactggaaa